(19) United States
(12) Patent Application Publication
(10) Pub. No.: US 2024/0277207 A1
(43) Pub. Date: Aug. 22, 2024

(54) SHOE CARE APPARATUS

(71) Applicant: Kohler Novita Ltd., Chungcheongnam-do (KR)

(72) Inventor: Wonkoo Cho, Chungcheongnam-do (KR)

(21) Appl. No.: 18/582,190

(22) Filed: Feb. 20, 2024

(30) Foreign Application Priority Data
Feb. 21, 2023 (KR) 10-2023-0022949

(51) Int. Cl. A47L 23/20

(52) U.S. Cl. CPC A47L 23/205

(57) ABSTRACT

A shoe care apparatus includes a body including at least one receiving space. The shoe care apparatus also includes an air suction and exhaust module disposed on a back side of the body. The shoe care apparatus also includes at least one shoe tray configured to be removed from a front side of the body or received in the at least one receiving space. The shoe care apparatus also includes at least one electrical board including an upper case and a lower case. The shoe care apparatus also includes a drying module disposed in an internal space formed by the upper case and the lower case. The shoe care apparatus also includes an air duct disposed in the internal space formed by the upper case and the lower case. The shoe care apparatus also includes at least one air supply hole disposed on the lower case.

### CROSS REFERENCE TO RELATED APPLICATION

This application claims the benefit of priority to: Korean Patent Application No. 10-2023-0022949 filed in the Korean Intellectual Property Office on Feb. 21, 2023, which is hereby incorporated by reference in its entirety.

### FIELD

The present disclosure relates to a shoe care apparatus, and more particularly the present disclosure relates to a shoe care apparatus having an improved air duct structure to accelerate an air flow for drying and/or deodorizing shoes.

### BACKGROUND

In general, when shoes get wet due to washing, going out, sweating, etc., the shoes are mainly dried by means of waiting for natural drying, using a hair dryer, or the like for forced drying.

However, in the natural drying or the forced drying using a hair dryer as described above, hot air cannot be evenly circulated in the interiors of the shoes, so that the interiors of the shoes cannot be completely dried. In particular, because the shoes are simply dried, there is a limitation in eliminating bacteria or viruses growing in the shoes.

In this regard, the granted Korean Utility Model Publication No. 20-0292366 discloses “shoe deodorization and sterilization device.”

Specifically, in the earlier granted Korean utility model patent, the ultraviolet and ozone sterilization effect is achieved by opening a door for opening or closing a space in a case, placing a shoe on a support component, inserting an ozone lamp into the shoe, closing the door, and operating an operation panel arranged on an outer surface of the door to apply a driving power to the ozone lamp under the control of a controller.

However, in the earlier Korean granted utility model patent, the sterilization structure is complex, and there is no additional drying assembly for drying the entire internal surface of the shoe, so that it is impossible to effectively inhibit bacterial reproduction under conditions such as moisture.

### SUMMARY

### Technical Problems to be Solved

The technical problem to be solved by the present disclosure is to provide a shoe care apparatus, which has an improved air duct structure to accelerate an air flow for drying and/or deodorizing shoes and also elaborately and evenly dries the entire internal surface of each of the shoes.

### Technical Solution to the Technical Problems

In an embodiment, the present disclosure provides a shoe care apparatus. The shore care apparatus includes a body, which has receiving spaces and is provided with an air suction and exhaust module formed on a back side. The shore care apparatus also includes shoe trays, which are removable from a front side of the body or receivable in one or more of the receiving spaces. The receiving spaces are formed by an electrical board, and the electrical board is a combined structure of an upper case and a lower case for accommodating a drying module. The shore care apparatus also includes an air duct formed inside the upper case and the lower case. The shore care apparatus also includes air supply holes formed in the lower case. The air supply holes are in communication with an outflow end of the air duct and supply dry air to the internal space.

In another embodiment, the receiving space includes a single first receiving space or includes a first receiving space and a second receiving space formed by division by the electrical board. The electrical board includes a first electrical board formed on a top side of the first receiving space or a first electrical board formed on the top side of the first receiving space and a second electrical board formed between the first receiving space and the second receiving space.

In another embodiment, the air duct is in the form of a Y-shaped bent portion formed by combination of the upper case and a partition formed in the lower case. One end of the Y-shaped bent portion is in communication with the air supply holes.

In another embodiment, an air care module is formed inside the air duct.

In another embodiment, an air suction port is formed in the lower case, and air sucked into the receiving space by the air suction and exhaust module is guided to the drying module by means of the air suction port.

In another embodiment, the shoe tray accommodated in the first receiving space or the shoe tray accommodated in the second receiving space is formed with an air nozzle assembly having an air intake port and an air spray port. The air intake port is formed at an upper end of the air nozzle assembly, so as to be in communication with the air supply holes when the shoe tray is enclosed in the first receiving space or the second receiving space. The air spray port is formed at a lower end of the air nozzle assembly, so as to spray the dry air or conditioned air flowing in through the air intake port.

In another embodiment, the air nozzle assembly includes a fixed portion fixed to a bottom side of the shoe tray and includes a tube portion having a bilaterally symmetrical structure at an upper end of the fixed portion. The air intake port is formed at an upper end of the tube portion, and the air spray port is formed at a lower end of the tube portion.

### Effects of the Present Disclosure

As described above, according to the present disclosure, an air duct structure is improved to accelerate an air flow for drying and/or deodorizing shoes, and the entire internal surface of each of the shoes is elaborately and evenly dried/deodorized, so as to improve the satisfaction of consumers.

The effects of the present disclosure are not limited to the above effects, and other effects not mentioned can be clearly understood by those having ordinary skill in the art from the present disclosure.

### BRIEF DESCRIPTION OF THE FIGURES

FIG. 1 is a structural perspective view of a shoe care apparatus according to an embodiment of the present disclosure.

FIG. 2 is a side cross-sectional view of the shoe care apparatus according to the embodiment of the present disclosure.

FIG. 3 is a perspective view of a combined structure of an upper case and a lower case of an electrical board according to the embodiment of the present disclosure.

FIG. 4 is an exploded view of the upper case and the lower case of the electrical board according to the embodiment of the present disclosure.

FIG. 5 is a schematic plan view of the lower case of the electrical board according to the embodiment of the present disclosure.

FIG. 6 is a flow chart of a shoe care method by using a shoe care apparatus according to an embodiment of the present disclosure.

### DETAILED DESCRIPTION

The advantages and features of the present disclosure and the methods for achieving the same should become more apparent with reference to the embodiments described in detail below together with the drawings. However, the embodiments of the present disclosure are not limited to the embodiments disclosed below but can be implemented in various forms. The embodiments are only intended to completely disclose the present disclosure and fully convey the scope of the present disclosure to those having ordinary skill in the art to which the present disclosure pertains. The scope of the present disclosure should be defined by the scope of the claims.

The terms used in the present disclosure are merely used to explain the embodiments, rather than limit the present disclosure. In the present disclosure, the singular form includes the plural form unless specified in the context.

In the present disclosure, it should be understood that the terms, such as “include” or “have”, are used to specify the existence of features, numbers, steps, actions, structural elements, components, or the combinations thereof recited in the present disclosure. The terms do not exclude the existence or additional possibility of one or more other features, numbers, steps, actions, structural elements, components, or the combinations thereof.

Furthermore, the embodiments described in the present disclosure are explained with reference to the exemplary cross-sectional view and/or top view of the present disclosure. Therefore, the embodiments of the present disclosure are not limited to the specific forms shown but include necessary form modifications. For example, an area represented by a right angle may have a circular arc shape or a shape having a specified curvature. Therefore, the area shown in the figure has schematic properties, and the shape of the area shown in the figure is to illustrate the specific shape of the area of a device, rather than limit the scope of the present disclosure.

Throughout the present disclosure, the same reference numerals represent the same or equivalent structural elements. Therefore, even if the reference numbers are not mentioned or explained in the corresponding drawings, the reference numbers may be explained with reference to the same reference numbers or similar reference numbers in other drawings. In addition, even if the reference numbers are not shown, they may be explained with reference to other drawings.

The embodiments of the present disclosure are described below with reference to the drawings.

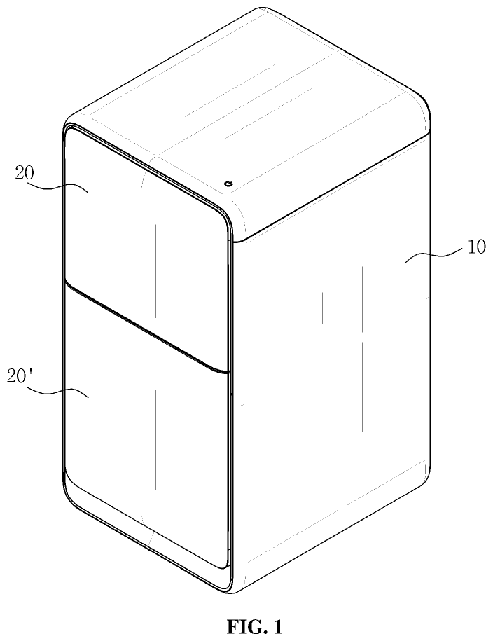

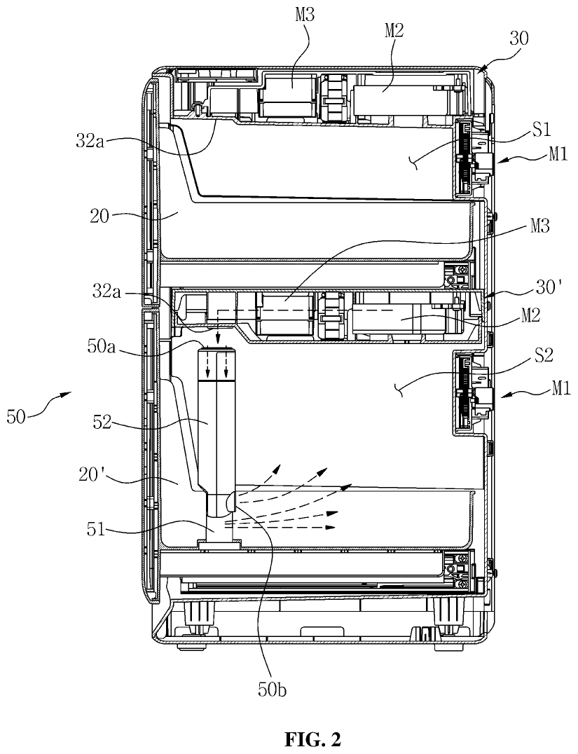

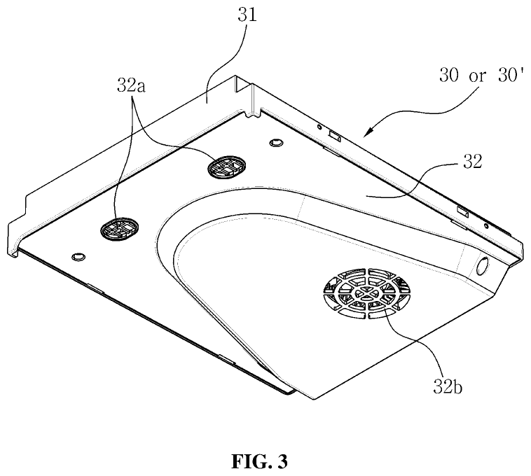

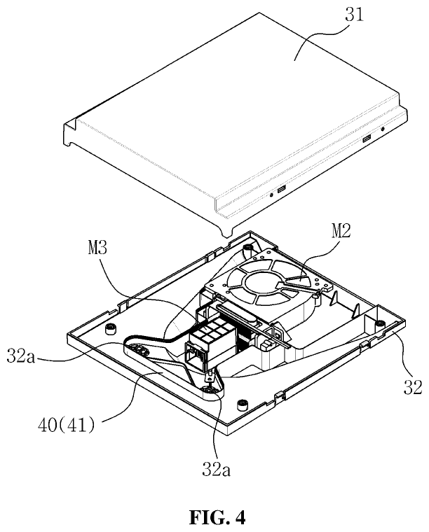

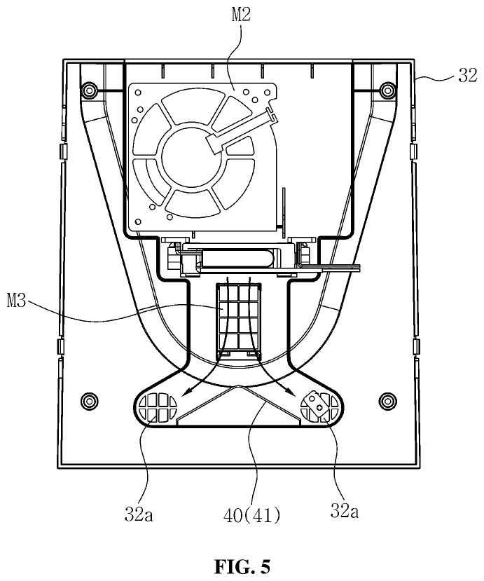

FIG. 1 is a structural perspective view of a shoe care apparatus according to an embodiment of the present disclosure. FIG. 2 is a side cross-sectional view of the shoe care apparatus according to the embodiment of the present disclosure. FIG. 3 is a perspective view of a combined structure of an upper case and a lower case of an electrical board according to the embodiment of the present disclosure. FIG. 4 is an exploded view of the upper case and the lower case of the electrical board according to the embodiment of the present disclosure. FIG. 5 is a schematic plan view of the lower case of the electrical board according to the embodiment of the present disclosure.

Referring to FIGS. 1-5, a shoe care apparatus according to an embodiment of the present disclosure comprises an interior of a body 10, which is divided into a first receiving space S1 and a second receiving space S2. The shoe care apparatus is used for caring shoes under the control of a control portion (e.g., a controller or a processor) and may include a body 10, shoe trays 20, 20′, a first electrical board 30, a second electrical board 30′, an air duct 40, and an air nozzle assembly 50.

The plurality of shoe trays 20, 20′ separated by the second electrical board 30′ are respectively accommodated in the first receiving space S1 and the second receiving space S2 formed in the body 10. Air suction and exhaust modules M1 configured to suck, suction, draw, pull, extract, inhale, or the like external air and exhausting internal air are respectively formed on an upper portion and a lower portion of a back side of the body 10. The air suction and exhaust module M1 is a combined structure of an air suction fan, an exhaust vent, an air exhaust louver, and a motor configured to drive the air exhaust louver to open or close.

The shoe trays 20, 20′ are drawer-type structures respectively accommodated in the first receiving space S1 and the second receiving space S2. The upper shoe tray 20 may have a first accommodation area, and the lower shoe tray 20′ may have a second accommodation area that is larger than the first accommodation area, such that the upper shoe tray 20 can accommodate small-sized shoes, and the lower shoe tray 20′ can accommodate small-sized and large-sized shoes.

The first electrical board 30 and the second electrical board 30′ may have the same structure. Each of the first electrical board 30 and the second electrical board 30′ may comprise a combined structure of an upper case 31 and a lower case 32 and may accommodate a drying module M2 comprising a combined structure of a heater (e.g., a PTC heater) and a fan. The first electrical board 30 may be formed on a top side of the first receiving space S1, and the second electrical board 30′ may be formed between the first receiving space S1 and the second receiving space S2 to serve as a partition.

In each of the first electrical board 30 and the second electrical board 30′ having the same structure, a pair of air supply holes 32a and an air suction port 32b may be formed in the lower case 32. The pair of air supply holes 32a are configured such that when the drying module M2 is driven, the dry air generated by the drying module M2 is supplied to the first receiving space S1 or the second receiving space S2. The air suction port 32b is configured such that when the drying module M1 is driven, the air flowing into the first receiving space S1 or the second receiving space S2 by means of the air suction and exhaust module M1 is sucked, suctioned, drawn, pulled, extracted, inhaled, or the like into the interiors of the upper case 31 and the lower case 32.

The air duct 40 is formed in the interior formed by the upper case 31 and the lower case 32 and has an outflow end in communication with the pair of air supply holes 32a.

Specifically, the air sucked, suctioned, drawn, pulled, extracted, inhaled, or the like through the air suction port 32b is heated when the drying module M2 is driven, and the air that is dried by means of the drying module M2 is discharged to the pair of air supply holes 32a through the air duct 40. The dried air may be supplied, through the air supply holes 32a, to the shoe tray(s) 20 and/or 20′ respectively accommodated in the first receiving space S1 and the second receiving space S2.

The air duct 40 is in the form of a Y-shaped bent portion formed by combining or connecting the upper case 31 and a partition 41 formed in the lower case 32, and one end of the Y-shaped bent portion, i.e., the outflow end, may be in communication with the pair of air supply holes 32a.

Furthermore, an air care module M3, which is a combined structure of a zeolite deodorant and an ionizer to serve as a deodorizing device, may be arranged inside the air duct 40, so that the dry air from the drying module M2 and/or the air conditioned by the air care module M3 may be guided to the air supply holes 32a through the air duct 40.

The air nozzle assembly 50 is formed on the shoe tray 20′ accommodated in the second receiving space S2 and includes a fixed portion 51 fixed to a bottom side of the shoe tray 20′. The air nozzle assembly 50 also includes a tube portion 52 having a bilaterally symmetrical structure at an upper end of the fixed portion 51. Air intake ports 50a are respectively formed at an upper end of the tube portion 52 having the bilaterally symmetrical structure. The air nozzle assembly 50 also includes air spray ports 50b are respectively formed at a lower end of the tube portion 52.

Specifically, the pair of air intake ports 50a are formed at the upper end of the tube portion 52 so as to be respectively in communication with the pair of air supply holes 32a when the shoe tray 20′ is enclosed and accommodated in the second receiving space S2. The pair of air spray ports 50b are formed at the lower end of the tube portion 52, such that the dry air or conditioned air flowing into the air intake ports 50a are respectively sprayed into the shoes accommodated in the shoe tray 20′.

As described above, as shown in FIGS. 1-5, during use of the shoe care apparatus according to the embodiment of the present disclosure, firstly, the shoe tray(s) 20 and/or 20′ are/is removed from the first receiving space S1 and/or the second receiving space S2 formed in the body 10, and then the shoes are placed in the removed shoe tray(s) 20 and/or 20′.

When the shoes are placed in the shoe tray 20′ having the second accommodation area, because the shoe tray 20′ is provided with the tube portion 52 that is bilaterally symmetrical with respect to the fixed portion 51 of the air nozzle assembly 50, the air spray ports 50b formed at the lower end of the tube portion 52 face the shoes placed therein.

After that, when the shoe tray(s) 20 and/or 20′ are/is respectively received in the first receiving space S1 and/or the second receiving space S2, the air intake ports 50a formed at the upper end of the bilaterally symmetrical tube portion 52 of the air nozzle assembly 50 are in communication with the air supply holes 32a formed at the lower case 32 of the second electrical board 30′.

Then, when the drying module M2 formed in the first electrical board 30 on the top side of the first receiving space S1 and/or the drying module M2 formed in the second electrical board 30′ as a partition structure for separating the first receiving space S1 and the second receiving space S2 are/is driven, the dry air heated when the drying module M2 is driven is guided by the air duct 40 to pass through the air supply holes 32a and the air intake ports 50a, which form a communication structure. The air flow is guided to the air spray ports 50b formed at the lower end of the tube portion 52, so that the dry air is sprayed through the air spray ports 50b and into the interior of the shoe that is placed in the shoe tray 20′ having the second accommodation area, and the interior of the shoe can be dried.

For the shoe placed in the shoe tray 20 having the first accommodation area, the dry air is sprayed into the first receiving space S1 through the air supply holes 32a of the lower case 32 of the first electrical board 30, so that the shoe placed in the shoe tray 20 can be dried.

Furthermore, as described above, the air that is dried when the drying module M2 is driven is sprayed. The air care module M3 is formed inside the air duct 40. When the air care module M3 is selectively driven, the dried air and the air conditioned by the air care module M3 are sprayed to the shoe tray(s) 20 and/or 20′ and thus deodorize the shoes.

In this case, the air care module M3 and the drying module M2 may be both driven. However, even if the drying module M2 is not operated, the interior of the shoe accommodated in the shoe tray(s) 20 and/or 20′ can also be deodorized when the air care module M3 is driven.

Furthermore, as described above, after the shoes are dried and deodorized, hot air remains in the first receiving space S1 and/or the second receiving space S2 in the body 10, and the air suction and exhaust module M1 is driven to discharge the hot air to the outside.

Furthermore, with reference to FIGS. 1-5, the internal space of the body 10 is divided into the first receiving space S1 and the second receiving space S2 by the second electrical board 30′, and the first electrical board 30 and the second electrical board 30′ having the same structure. The air suction and exhaust modules M1, the drying modules M2, and the air care modules M3 are respectively formed in the first receiving space S1 and the second receiving space S2. However, there may be only a single first receiving space S1 formed in the internal space of the body 10.

Specifically, in the case where a single first receiving space S1 is formed inside the body 10, the first electrical board 30 for arranging the drying module M2 and the air care module M3 may be formed on the top side of the first receiving space S1. The air nozzle assembly 50 may be formed on the shoe tray 20 accommodated in the first receiving space S1. Thus, there may be only a single air suction and exhaust module M1 formed on the back side of the body 10. Such a structure operates in the same way as that in FIGS. 1-5 of the embodiment of the present disclosure, so the detailed description thereof is omitted herein.

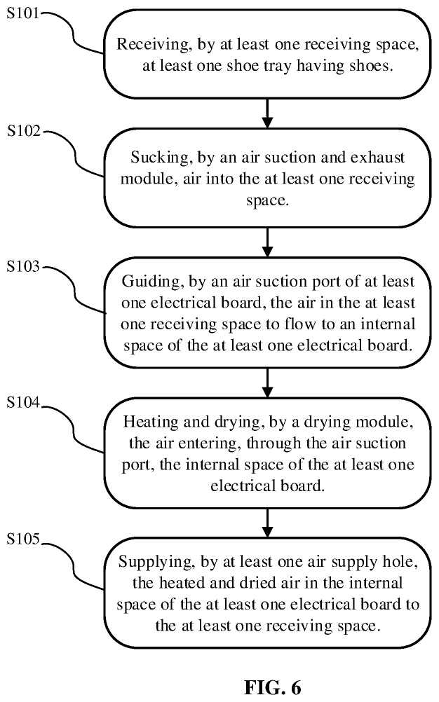

FIG. 6 is a flow chart of a shoe care method by using a shoe care apparatus according to an embodiment of the present disclosure. The shoe care apparatus used by the method may be the shoe care apparatus according to any of the foregoing embodiments and may be configured to perform an operation, function, or the like as described in the present disclosure. The detailed descriptions regarding the shoe care apparatus are incorporated herein.

At act S101, the at least one receiving space S1, S2 receives the at least one shoe tray 20, 20′ having shoes.

Specifically, as described above, the plurality of shoe trays 20, 20′ separated by the second electrical board 30′ are respectively accommodated in the first receiving space S1 and the second receiving space S2 formed in the body 10. The shoe trays 20, 20′ are drawer-type structures respectively accommodated in the first receiving space S1 and the second receiving space S2. The upper shoe tray 20 may have a first accommodation area, and the lower shoe tray 20′ may have a second accommodation area that is larger than the first accommodation area, such that the upper shoe tray 20 can accommodate small-sized shoes, and the lower shoe tray 20′ can accommodate small-sized and large-sized shoes.

In another embodiment, a single first receiving space S1 is formed inside the body 10. Such a structure operates in the same way as that in FIGS. 1-5 of the embodiment of the present disclosure.

At act S102, the air suction and exhaust module M1 sucks the air into the at least one receiving space S1, S2.

Specifically, as described above, the air suction and exhaust modules M1 configured to suck, suction, draw, pull, extract, inhale, or the like external air and exhausting internal air are respectively formed on an upper portion and a lower portion of a back side of the body 10. The air suction and exhaust module M1 is a combined structure of an air suction fan, an exhaust vent, an air exhaust louver, and a motor configured to drive the air exhaust louver to open or close. When the drying module M1 is driven, the air flows into the first receiving space S1 or the second receiving space S2.

In another embodiment, there may be only a single air suction and exhaust module M1 formed on the back side of the body 10. Such a structure operates in the same way as that in FIGS. 1-5 of the embodiment of the present disclosure.

At act S103, the air suction port 32b of the at least one electrical board 30, 30′ guides the air in the at least one receiving space S1, S2 to flow to the internal space of the at least one electrical board 30, 30′.

Specifically, as described above, in each of the first electrical board 30 and the second electrical board 30′ having the same structure, the air suction port 32b may be formed in the lower case 32. The air suction port 32b is configured such that when the drying module M1 is driven, the air flowing into the first receiving space S1 or the second receiving space S2 by means of the air suction and exhaust module M1 is sucked, suctioned, drawn, pulled, extracted, inhaled, or the like into the interiors of the upper case 31 and the lower case 32.

In another embodiment, in the case where a single first receiving space S1 is formed inside the body 10, the first electrical board 30 for arranging the drying module may be formed on the top side of the first receiving space S1.

At act S104, the drying module M2 heats and dries the air entering, through the air suction port 32b, the internal space of the at least one electrical board 30, 30′.

Specifically, as described above, each of the first electrical board 30 and the second electrical board 30′ may comprise the drying module M2 comprising a combined structure of a heater (e.g., a PTC heater) and a fan. The drying module M2 is formed in the first electrical board 30 on the top side of the first receiving space S1 and/or the drying module M2 is formed in the second electrical board 30′ as a partition structure for separating the first receiving space S1 and the second receiving space S2. The air sucked, suctioned, drawn, pulled, extracted, inhaled, or the like through the air suction port 32b is heated and dried when the drying module M2 is driven.

At act S105, the at least one air supply hole 32a supplies the heated and dried air in the internal space of the at least one electrical board 30, 30′ to the at least one receiving space S1, S2.

Specifically, as described above, the air that is heated and dried by means of the drying module M2 is discharged to the pair of air supply holes 32a through the air duct 40. The heated and dried air may be supplied, through the air supply holes 32a, to the shoe tray(s) 20 and/or 20′ respectively accommodated in the first receiving space S1 and the second receiving space S2. Specifically, the heated and dried air when the drying module M2 is driven is guided by the air duct 40 to pass through the air supply holes 32a and the air intake ports 50a, which form a communication structure. The air flow is guided to the air spray ports 50b formed at the lower end of the tube portion 52, so that the heated and dried air is sprayed through the air spray ports 50b and into the interior of the shoe that is placed in the shoe tray 20′ having the second accommodation area, and the interior of the shoe can be dried.

In another embodiment, in the case where a single first receiving space S1 is formed inside the body 10, the air nozzle assembly 50 may be formed on the shoe tray 20 accommodated in the first receiving space S1.

The shoe care apparatus and method of the present disclosure have been explained above with reference to the drawings. However, the above descriptions are only intended to illustrate the embodiments of the present disclosure, rather than limit the present disclosure.

Therefore, the present disclosure is not limited to the above specific embodiments, and those having ordinary skill in the art to which the present disclosure pertains may implement various modifications without exceeding the gist of the present disclosure as claimed in the claims, and these modifications are also within the scope of the present disclosure.

List of reference signs

| | |
|---|---|
| 10: body; | 20, 20′: shoe tray; |
| 30, 30′: electrical board; | 31: upper case; |
| 32: lower case; | 32a: air supply hole; |
| 32b: air suction port; | 40: air duct; |
| 41: partition; | 50: air nozzle assembly; |
| 50a: air intake port; | 50b: air spray port; |
| 51: fixed portion; | 52: tube portion; |
| M1: air suction and exhaust module; | M2: drying module; |
| M3: air care module; | S1, S2: receiving spaces. |

1-7. (canceled)

8. A shoe care apparatus, comprising:
a body comprising at least one receiving space;
an air suction and exhaust module disposed on a back side of the body;
at least one shoe tray configured to be removed from a front side of the body or received in the at least one receiving space, the at least one shoe tray configured to receive shoes;
at least one electrical board comprising an upper case and a lower case;
a drying module disposed in an internal space formed by the upper case and the lower case;
an air duct disposed in the internal space formed by the upper case and the lower case; and
at least one air supply hole disposed on the lower case and in communication with the air duct.

9. The shoe care apparatus according to claim 8,
wherein the at least one receiving space comprises a single receiving space, and
wherein the at least one electrical board comprises a single electrical board disposed on a top side of the single receiving space.

10. The shoe care apparatus according to claim 9,
wherein the at least one shoe tray comprises a single shoe tray configured to be received in the single receiving space, and
wherein the shoe care apparatus further comprises an air nozzle assembly disposed on the single shoe tray.

11. The shoe care apparatus according to claim 8,
wherein the at least one receiving space comprises a first receiving space and a second receiving space,
wherein the at least one electrical board comprises a first electrical board disposed on a top side of the first receiving space and a second electrical board disposed on a top side of the second receiving space, and
wherein the second electrical board divides an internal space of the body into the first receiving space and the second receiving space.

12. The shoe care apparatus according to claim 11,
wherein the at least one shoe tray comprises a first shoe tray configured to be received in the first receiving space and a second shoe tray configured to be received in the second receiving space, and
wherein the shoe care apparatus further comprises an air nozzle assembly disposed on the second shoe tray.

13. The shoe care apparatus according to claim 8,
wherein the at least one air supply hole comprises two air supply holes, and
wherein the air duct comprises a Y-shaped bent portion formed by connecting the upper case and a partition formed in the lower case, and
wherein one end of the Y-shaped bent portion is in communication with the two air supply holes.

14. The shoe care apparatus according to claim 8, further comprising:
an air care module disposed in the air duct and configured to deodorize air in the internal space formed by the upper case and the lower case.

15. The shoe care apparatus according to claim 8, further comprising:
an air suction port disposed in the lower case,
wherein the air suction and exhaust module is configured to suck air into the at least one receiving space, and
wherein the air suction port is configured to guide air in the at least one receiving space to flow to the drying module.

16. The shoe care apparatus according to claim 8, further comprising:
an air nozzle assembly disposed on the at least one shoe tray, the air nozzle assembly comprising:
at least one air intake port disposed at an upper end of the air nozzle assembly and in communication with the at least one air supply hole when the at least one shoe tray is received in the at least one receiving space; and
at least one air spray port disposed at a lower end of the air nozzle assembly and configured to spray air, entering the at least one air intake port, into the shoes received in the at least one shoe tray.

17. The shoe care apparatus according to claim 16,
wherein the air nozzle assembly further comprises:
a fixed portion fixed to a bottom side of the at least one shoe tray; and
a tube portion having a bilaterally symmetrical structure and disposed at an upper end of the fixed portion, and
wherein the at least one air intake port is disposed at an upper end of the tube portion, and the at least one air spray port is disposed at a lower end of the tube portion.

18. The shoe care apparatus according to claim 17,
wherein the at least one air intake port comprises two air intake ports disposed at the upper end of the tube portion, and
wherein the at least one air spray port comprises two air spray ports disposed at the lower end of the tube portion.

19. The shoe care apparatus according to claim 8,
wherein the air suction and exhaust module is configured to suck air into the at least one receiving space,
wherein the shoe care apparatus further comprises an air suction port disposed in the lower case and configured to guide the air to flow from the at least one receiving space to the internal space formed by the upper case and the lower case,
wherein the drying module is configured to heat and dry the air entering, through the air suction port, the internal space formed by the upper case and the lower case,
wherein the at least one air supply hole is configured to supply the heated and dried air in the internal space formed by the upper case and the lower case to the at least one receiving space,
wherein the air suction and exhaust module is further configured to discharge the heated and dried air in the at least one receiving space into outside.

20. A shoe care method by using a shoe care apparatus, the method comprising:
receiving, by at least one receiving space, at least one shoe tray having shoes;
sucking, by an air suction and exhaust module, air into the at least one receiving space;
guiding, by an air suction port of at least one electrical board, the air in the at least one receiving space to flow to an internal space of the at least one electrical board;
heating and drying, by a drying module, the air entering, through the air suction port, the internal space of the at least one electrical board; and
supplying, by at least one air supply hole, the heated and dried air in the internal space of the at least one electrical board to the at least one receiving space.

21. The shoe care method according to claim 20, further comprising:
deodorizing, by an air care module disposed in an air duct of the at least one electrical board, the air entering the internal space of the at least one electrical board.

22. The shoe care method according to claim 20, further comprising:
discharging, by the air suction and exhaust module, the heated and dried air in the at least one receiving space into outside.

23. The shoe care method according to claim 20,
wherein the at least one air supply hole comprises two air supply holes,
wherein the shoe care method further comprises:
guiding, by a Y-shaped air duct of the at least one electrical board, the air to flow from the air suction port to the two air supply holes.

24. The shoe care method according to claim 20, further comprising:
spraying, by an air nozzle assembly disposed on the at least one shoe tray, the air from the at least one air supply hole into the shoes received in the at least one shoe tray.

25. The shoe care method according to claim 24,
wherein spraying, by the air nozzle assembly disposed on the at least one shoe tray, the air from the at least one air supply hole into the shoes received in the at least one shoe tray comprises:
guiding, by at least one air intake port disposed at an upper end of a tube portion of the air nozzle assembly, the air to flow from the at least one air supply hole to the tube portion; and
guiding, by at least one air spray port disposed at a lower end of the tube portion of the air nozzle assembly, the air to flow from the tube portion to the at least one shoe tray.

26. The shoe care method according to claim 25,
wherein the at least one air intake port comprises two air intake ports disposed at the upper end of the tube portion,
wherein guiding, by the at least one air intake port disposed at the upper end of the tube portion of the air nozzle assembly, the air to flow from the at least one air supply hole to the tube portion comprises:
guiding, by the two air intake ports, the air to flow from the at least one air supply hole to the tube portion having a bilaterally symmetrical structure.

27. The shoe care method according to claim 26,
wherein the at least one air spray port comprises two air spray ports disposed at the lower end of the tube portion, and
wherein guiding, by the at least one air spray port disposed at the lower end of the tube portion of the air nozzle assembly, the air to flow from the tube portion to the at least one shoe tray comprises:
guiding, by the two air spray ports, the air to flow from the tube portion having the bilaterally symmetrical structure to the at least one shoe tray.
